(19) United States
(12) Patent Application Publication
(10) Pub. No.: US 2015/0017252 A1
(43) Pub. Date: Jan. 15, 2015

(54) SUPPLEMENT AND MEDICATION CULTIVATED PLANT DELIVERY SYSTEM

(71) Applicants: Wayne Garland, Gainesville, FL (US); Allen Mark Jones, Imperial Beach, CA (US); G. Douglas Hayden, Richmond, VA (US)

(72) Inventors: Wayne Garland, Gainesville, FL (US); Allen Mark Jones, Imperial Beach, CA (US); G. Douglas Hayden, Richmond, VA (US)

(21) Appl. No.: 14/330,036

(22) Filed: Jul. 14, 2014

Related U.S. Application Data
(60) Provisional application No. 61/957,892, filed on Jul. 15, 2013.

(51) Int. Cl. A23L 1/29; A01G 31/00; A61K 36/00

(52) U.S. Cl. CPC A23L 1/293; A61K 36/00; A01G 31/00; A23V 2002/00; A01G 2031/006
USPC 424/538; 424/725; 424/520; 426/2; 47/62 N

(57) ABSTRACT

The embodiments disclose a supplement and medication cultivated plant delivery system including using a cultivation system for cultivating integrated enriched food products including fruits, produce and aquatic species and animal feeds, controlling and testing the cultivation system environment including an aqua cultivation media, incorporating a controlled feed system to raise aquatic species and animals using integrated enriched food products feeds, harvesting cultivated integrated enriched food products and cultivated integrated enriched food products feed raised aquatic species and animals, delivering the cultivated integrated enriched food stuffs to markets for providing alternative cultivated integrated enriched food stuffs to prevent and treat health conditions on a long-term sustainable basis, and empowering people to improve their health conditions while simply eating cultivated integrated enriched food stuffs in their daily regimen of eating.

### CROSS-REFERENCE TO RELATED APPLICATIONS

This application is based on U.S. Provisional Patent Application Ser. No. 61/957,892 filed Jul. 15, 2013, entitled “A Supplement and Medication Cultivated Plant Delivery System”, by First Named Inventor Dr. Wayne Garland.

### BACKGROUND

The medical profession has long been frustrated by the numbers of patients with a lack of diligence in taking medication as prescribed to them. Additionally the use of supplements by patients to improve their health is met with the same inconsistency of dosing. Poor patient regimen in taking medications and supplements leads to prolonged recovery and in some cases a progressive escalation of the condition being treated and added health care costs.

### BRIEF DESCRIPTION OF THE DRAWINGS

FIG. 1 shows a block diagram of an overview of a supplement and medication cultivated plant delivery system of one embodiment.

FIG. 2 shows a block diagram of an overview flow chart of a supplement and medication cultivated plant delivery system of one embodiment.

FIG. 3 shows a block diagram of an overview flow chart of cultivating any number and multiple types of plants of one embodiment.

FIG. 4 shows a block diagram of an overview flow chart of cultivated integrated enriched food products feed of one embodiment.

FIG. 5 shows a block diagram of an overview flow chart of harvesting cultivated integrated enriched food products of one embodiment.

FIG. 6 shows a block diagram of an overview flow chart of combining the daily dietary needs with the beneficial aspects of dietary supplements and medicinal treatment of one embodiment.

FIG. 7 shows for illustrative purposes only an example of hydroponic growing container with young plant and root system of one embodiment.

FIG. 8 shows for illustrative purposes only an example of developed “fruit” contains supplemental vitamins and minerals of one embodiment.

FIG. 9A shows a block diagram of controlling the aqua cultivation media composition of one embodiment.

FIG. 9B shows a block diagram of testing the aqua cultivation media composition of one embodiment.

FIG. 10 shows a block diagram of examples chemical elements and/or compounds of those chemical elements additives of one embodiment.

FIG. 11A shows a block diagram of examples of one or more vitamin liquid additives of one embodiment.

FIG. 11B shows a block diagram of examples of one or more liquid concentrates of organic vegetables additives of one embodiment.

FIG. 11C shows a block diagram of examples of one or more liquid concentrates of sea vegetables additives of one embodiment.

FIG. 11D shows a block diagram of examples of one or more plant root absorption stimulant additives of one embodiment.

FIG. 11E shows a block diagram of examples of one or more liquid concentrates of organic herbal compounds additives of one embodiment.

FIG. 12A shows a block diagram of examples of medicinal herbs of one embodiment.

FIG. 12B shows a block diagram of a continuation of examples of medicinal herbs of one embodiment.

FIG. 13A shows a block diagram of examples of culinary herbs and spices of one embodiment.

FIG. 13B shows a block diagram of a continuation of examples of culinary herbs and spices of one embodiment.

FIG. 14 shows a block diagram of examples of grains of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview:

It should be noted that the descriptions that follow, for example, in terms of a supplement and medication cultivated plant delivery system is described for illustrative purposes and the underlying system can apply to any number and multiple types of plants. In one embodiment of the present invention, the supplement and medication cultivated plant delivery system can be configured using vegetable and fruit plants grown hydroponically. The supplement and medication cultivated plant delivery system can be configured to include growing supplement and medication cultivated plant feed and can be configured to include a feed system to raise fish, mollusks, crustaceans, poultry and livestock animals using the present invention.

The cultivation of plants for human consumption and as feed for various species used as food by humans has continued through most of millennium of humans. Medicinal plants have also been a part of early medical treatment for various health conditions as remedies. Modern pharmaceutical products tend to be pills or injections of medications. From the time we are children there is an inherent dislike of taking pills or fear associated with facing a needle for an injection. The cultural orientation appears to continue into adulthood as major numbers of adult patients will not follow doctor's orders when taking the prescribed numbers or frequencies of pills or self-injections.

Today the trend of seeking a healthier life style is growing. Organically raised fruits, produce, poultry and other foodstuffs are becoming more available. But it is far from reaching the masses in part due to cost and availability. Concern is at a high in the population, particularly the baby boomers and political agencies about the cost of health care. The recent studies have shown the burdensome economics of the current health care delivery systems using current pharmaceuticals.

But, people still eat daily. The present invention combines the daily dietary needs with the beneficial aspects of dietary supplements and medicinal treatment. The supplement and medication cultivated plant delivery system cultivates fruits, produce and other plants to include additional supplemental vitamins and minerals to provide a level of treatment for various conditions. In addition the supplement and medication cultivated plant delivery system is used to raise feed products for use in raising fish, mollusks, crustaceans, poultry and livestock for human consumption providing additional dietary elements with integrated enriched supplemental vitamins and minerals to provide a level of treatment for various conditions.

Integrated enriched foodstuffs cultivated using the supplement and medication cultivated plant delivery system can be made available in markets. People can purchase the integrated enriched foodstuffs in uncooked (raw), pre-cooked and preprocessed food products. These can replace other food products that do not have the integrated enriched supplemental vitamins and minerals. The cost can be competitive with other food products currently available, so the people can improve their health conditions while simply eating their daily regimen of dietary needs without the dislikes and fear associated with taking medications. The cost to cultivate and delivery of the integrated enriched supplemental vitamins and minerals food products will be far less than current healthcare delivery systems.

Certain integrated enriched supplemental vitamins and minerals food products can be cultivated using specific supplemental vitamins and minerals to treat a specific condition or group of conditions and labeled as such. The cultivation can be controlled to stimulate plant root absorption of the specific supplemental vitamins and minerals. Testing of plants cultivated using the supplement and medication cultivated plant delivery system can be done during the growing cycle to monitor the levels of supplemental vitamins and minerals being absorbed by the plants. Testing can be performed at harvest to confirm the levels of the various supplemental vitamins and minerals to provide labeling data.

The cultivation systems used in the supplement and medication cultivated plant delivery system will not use pesticides and other harmful chemicals. The cultivation systems used in the supplement and medication cultivated plant delivery system may include the use of hydroponic gardening, aquaculture in tanks and controlled environmental pens for livestock. This will meet the desires of the populace for organically grown foodstuffs. In addition to eliminating the use of harmful chemicals it will provide the additional supplemental vitamins and minerals to prevent and possibly cure health conditions on a long-term sustainable basis. The public is accepting of the long term use of supplementing food products with supplemental vitamins and minerals including for example iodine in salt to prevent hypothyroidism, and vitamins A, B, C, D and proteins and dietary fibers as preventative and treatment for other conditions.

One advantage of the supplement and medication cultivated plant delivery system is that these supplemental vitamins and minerals are integrated into the growing of the fruits, produce, plants, fishes, mollusks, crustaceans, poultry and livestock not added during a food processing operation. In this way a person can for example prepare the raw uncooked integrated enriched food products in any way they desire including eating the produce and fruits raw for example in salads, fishes in a sashimi style and livestock in a beef tartare. The person may prepare the integrated enriched food products using various cooking techniques and fully replace the current food products available. The supplement and medication cultivated plant delivery system includes delivery of prepackaged prepared foods and meals using the integrated enriched supplemental vitamins and minerals food products. Through their normal eating habits a person can maintain their daily dietary regime and receive the supplemental vitamins, minerals and compounds to prevent and treat conditions while minimizing or eliminating the extra efforts, costs and scheduling of taking separate medications and supplements.

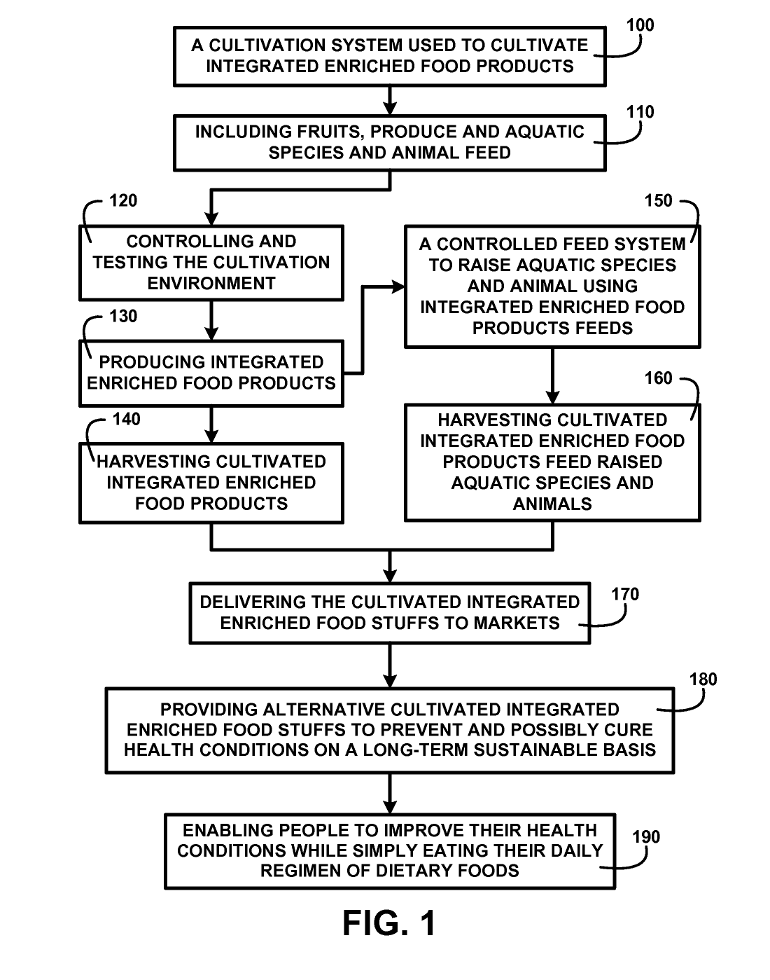

FIG. 1 shows a block diagram of an overview of a supplement and medication cultivated plant delivery system of one embodiment. FIG. 1 shows a cultivation system used to cultivate integrated enriched food products 100 including fruits, produce and aquatic species and animal feed 110. The cultivation system used to cultivate integrated enriched food products 100 includes controlling and testing the cultivation environment 120 and cultivated plants. Producing integrated enriched food products 130 include a controlled feed system to raise aquatic species and animal using integrated enriched food products feeds 150 of one embodiment.

Harvesting cultivated integrated enriched food products 140 and harvesting cultivated integrated enriched food products feed raised aquatic species and animals 160 produces food stuffs with supplemental vitamins and minerals. Post harvesting processes include delivering the cultivated integrated enriched food stuffs to markets 170 for providing alternative cultivated integrated enriched food stuffs to prevent and possibly cure health conditions on a long-term sustainable basis 180. Providing alternative cultivated integrated enriched food stuffs to prevent and possibly cure health conditions on a long-term sustainable basis 180 is enabling people to improve their health conditions while simply eating their daily regimen of dietary foods 190. The supplement and medication cultivated plant delivery system provides a viable alternative to conventional health care delivery systems, a solution to poor patient regimen in taking medications and supplements and a real way to reduce patient care health care costs of one embodiment.

### Detailed Description:

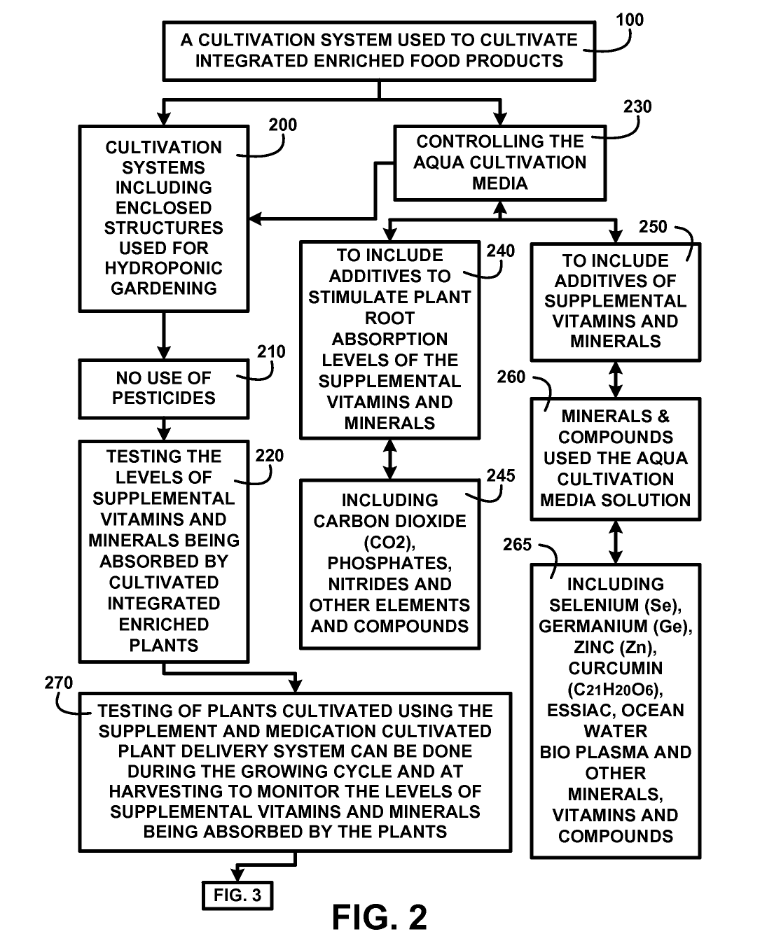

FIG. 2 shows a block diagram of an overview flow chart of a supplement and medication cultivated plant delivery system of one embodiment. FIG. 2 shows a cultivation system used to cultivate integrated enriched food products 100 including cultivation systems including enclosed structures used for hydroponic gardening 200. The cultivation system used to cultivate integrated enriched food products 100 when incorporated in hydroponic gardening includes controlling the aqua cultivation media 230 to include additives to stimulate plant root absorption levels of the supplemental vitamins and minerals 240. Additives to stimulate plant root absorption levels including carbon dioxide (co2), phosphates, nitrides and other elements and compounds 245 are used to enable plants to absorb nutrients and additives of supplemental vitamins and minerals 250 of one embodiment.

Minerals & compounds used the aqua cultivation media solution 260 including selenium (se), germanium (ge), zinc (zn), curcumin (c21h20o6), essiac, ocean water bio plasma and other minerals, vitamins and compounds 265 will be absorbed by the plants including the “fruit” wherein the “fruit” includes any edible part of the plant. The cultivation system used to cultivate integrated enriched food products 100 has no use of pesticides 210 of one embodiment.

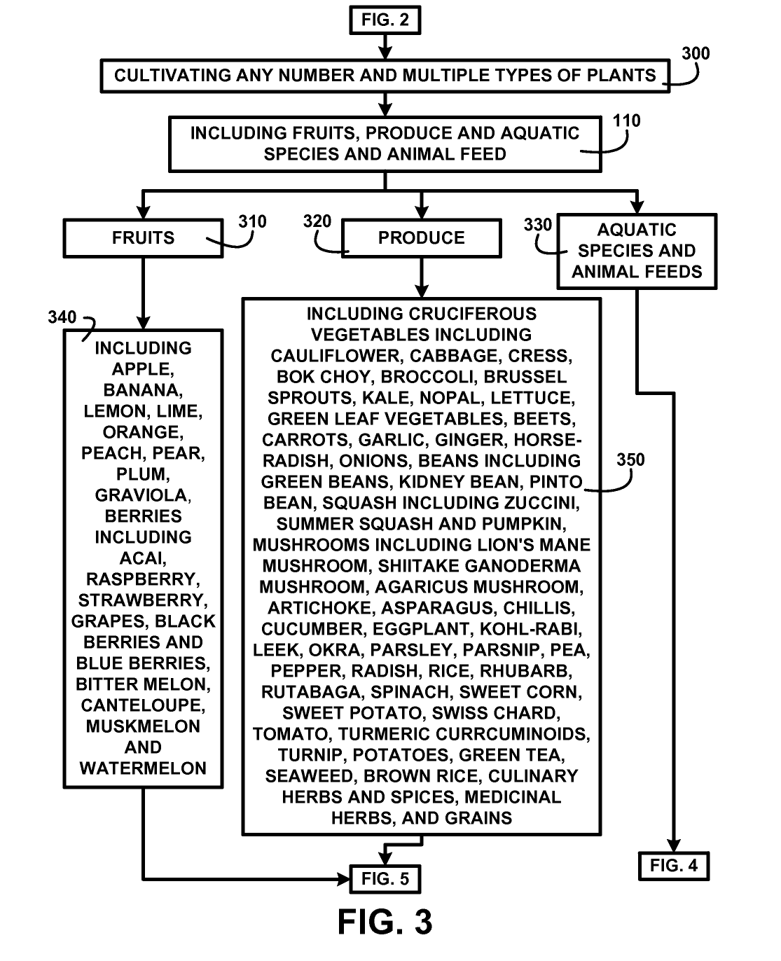

Testing the levels of supplemental vitamins and minerals being absorbed by cultivated plants 220 include qualitative analysis and quantitative analysis. Qualitative analysis is used to determine the elements and compounds present in a sample of cultivated integrated enriched plants. Quantitative analysis is used to determine the percentage of elements and compounds present in a sample of cultivated integrated enriched plants. The elements and compounds determined include the mineral, vitamin and compound additives infused into the aqua cultivation media thereby creating an enriched aqua cultivation media. Testing will include FDA nutrition facts value label testing. The testing will include using chemical analysis including spectrographic analysis to check levels of the supplemental vitamins and minerals absorbed by the various plant types. Testing of plants cultivated using the supplement and medication cultivated plant delivery system can be done during the growing cycle and at harvesting to monitor the levels of supplemental vitamins and minerals being absorbed by the plants 270. Processes include further descriptions in FIG. 3 of one embodiment.

### Cultivating Any Number and Multiple Types of Plants:

FIG. 3 shows a block diagram of an overview flow chart of cultivating any number and multiple types of plants of one embodiment. FIG. 3 shows continuing from FIG. 2 including cultivating any number and multiple types of plants 300 including fruits, produce and aquatic species and animal feed 110. The supplement and medication cultivated plant delivery system includes cultivation of fruits 310 including apple, banana, lemon, lime, orange, peach, pear, plum, graviola, berries including acai, raspberry, strawberry, black berries and blue berries, bitter melon, canteloupe, muskmelon and watermelon 340 of one embodiment.

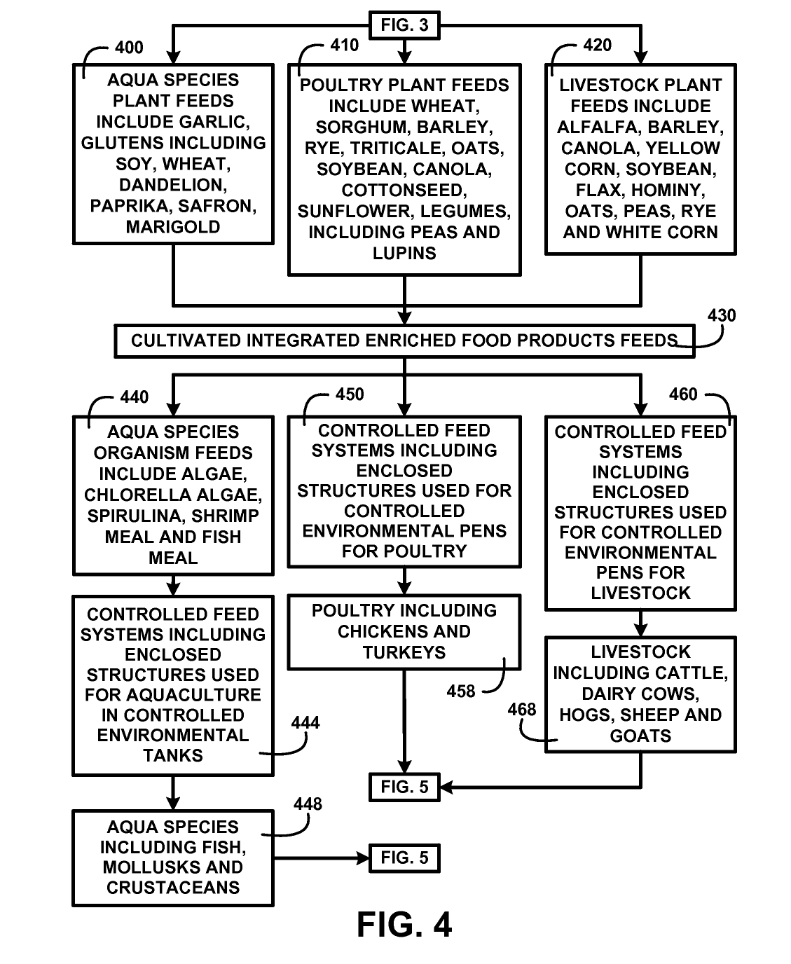

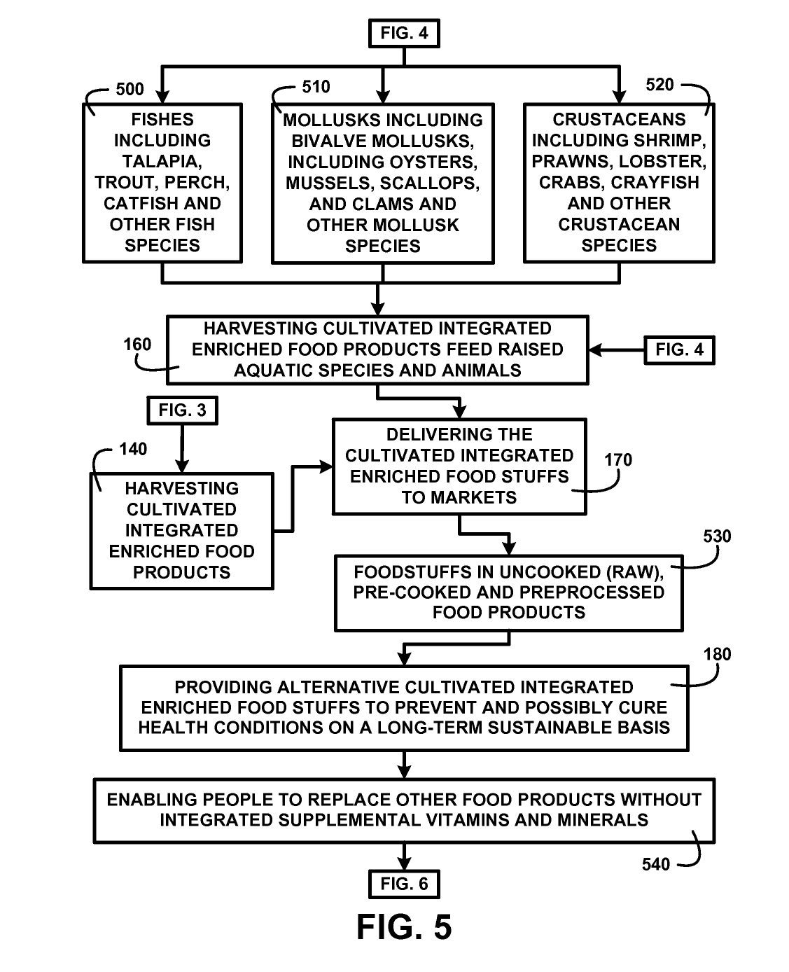

The supplement and medication cultivated plant delivery system includes cultivation of produce 320 including cruciferous vegetables including cauliflower, cabbage, cress, bok choy, broccoli, brussel sprouts, kale, lettuce and similar green leaf vegetables, beets, carrots, garlic, ginger, horse-radish, onions, beans including green beans, kidney bean, pinto bean, squash including zucchini, summer squash and pumpkin, mushrooms including lion's mane mushroom, shiitake ganoderma mushroom, agaricus mushroom, artichoke, asparagus, chillis, cucumber, eggplant, kohl-rabi, leek, okra, parsley, parsnip, pea, pepper, radish, rhubarb, rutabaga, spinach, sweet corn, sweet potato, swiss chard, tomato, turmeric currcuminoids, turnip, potatoes, green tea, seaweed and brown rice 350. These processes are further described in FIG. 5. The supplement and medication cultivated plant delivery system includes cultivation of aquatic species and animal feeds 330 as further described in FIG. 4 of one embodiment.

### Cultivated Integrated Enriched Food Products Feed:

FIG. 4 shows a block diagram of an overview flow chart of cultivated integrated enriched food products feed of one embodiment. FIG. 4 shows continuing from FIG. 3 aqua species plant feeds include garlic, glutens including soy, wheat, dandelion, paprika, saffron, marigold 400. Poultry plant feeds include wheat, sorghum, barley, rye, triticale, oats, soybean, canola, cottonseed, sunflower, legumes, including peas and lupins 410. Livestock plant feeds include alfalfa, barley, canola, yellow corn, soybean, flax, hominy, oats, peas, rye and white corn 420 of one embodiment.

Cultivated integrated enriched food products feeds 430 include aqua species organism feeds include algae, chlorella algae, spirulina, shrimp meal and fish meal 440. One embodiment of Controlled feed systems including enclosed structures used for aquaculture in controlled environmental tanks 444 can be used to raise aqua species including fish, mollusks and crustaceans 448 including feeding them the cultivated integrated enriched food products feeds 430 of one embodiment.

In another embodiment controlled feed systems including enclosed structures used for controlled environmental pens for poultry 450 to raise poultry including chickens and turkeys 458 including feeding them the cultivated integrated enriched food products feeds 430. Another embodiment includes controlled feed systems including enclosed structures used for controlled environmental pens for livestock 460 including cattle, dairy cows, hogs, sheep and goats 468 including feeding them the cultivated integrated enriched food products feeds 430. Descriptions of continuing processes are shown in FIG. 5 of one embodiment.

### Harvesting Cultivated Integrated Enriched Food Products:

FIG. 5 shows a block diagram of an overview flow chart of harvesting cultivated integrated enriched food products of one embodiment. FIG. 5 shows continuing from FIG. 4 the supplement and medication cultivated plant delivery system includes fishes including tilapia, trout, perch, catfish and other fish species 500, mollusks including bivalve mollusks, including oysters, mussels, scallops, and clams and other mollusk species 510 and crustaceans including shrimp, prawns, lobster, crabs, crayfish and other crustacean species 520 of one embodiment.

Also shown continuing from FIG. 4 is harvesting cultivated integrated enriched food products feed raised aquatic species and animals 160. Continued from FIG. 3 is harvesting cultivated integrated enriched food products 140. The harvesting is followed by packaging and delivering the cultivated integrated enriched food stuffs to markets 170. Foodstuffs in uncooked (raw), pre-cooked and preprocessed food products 530 can be processed, packaged and made available in markets and other outlet methods including purchasing online over the internet and by catalogue sales where orders can include telephone orders of one embodiment.

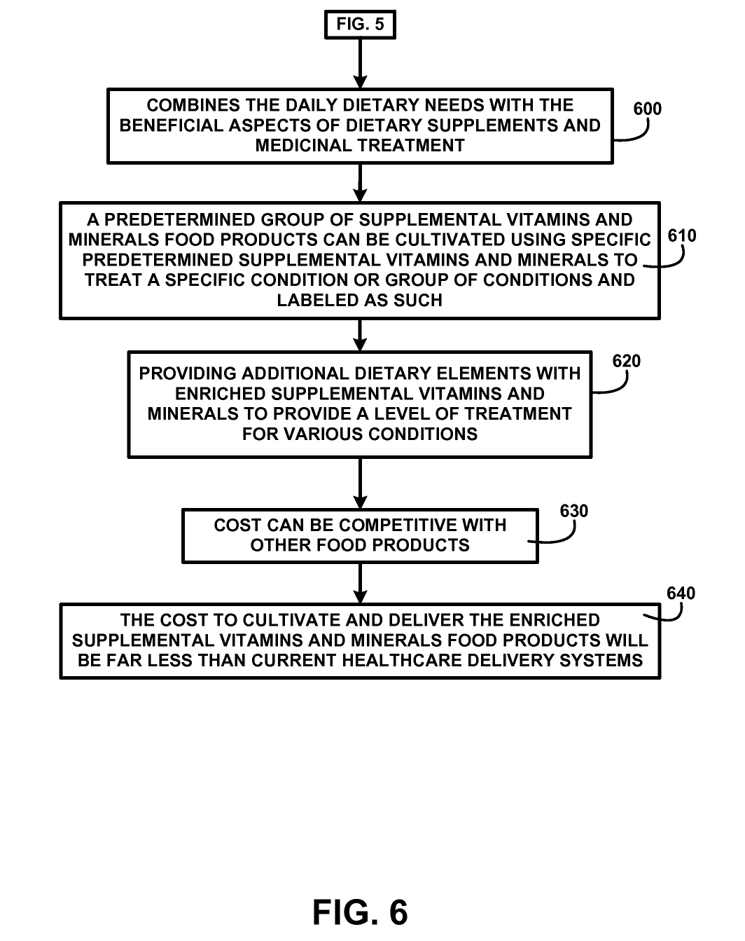

Providing alternative cultivated integrated enriched food stuffs to prevent and possibly cure health conditions on a long-term sustainable basis 180 is enabling people to replace other food products without integrated supplemental vitamins and minerals 540. Further descriptions of the supplement and medication cultivated plant delivery system are in FIG. 6 of one embodiment.

Combining Daily Dietary Needs with the Beneficial Aspects of Dietary Supplements and Medicinal Treatment:

FIG. 6 shows a block diagram of an overview flow chart of combining the daily dietary needs with the beneficial aspects of dietary supplements and medicinal treatment of one embodiment. FIG. 6 shows a continuation from FIG. 5 where the supplement and medication cultivated plant delivery system combines the daily dietary needs with the beneficial aspects of dietary supplements and medicinal treatment 600. Certain integrated enriched supplemental vitamins and minerals food products can be cultivated using specific supplemental vitamins and minerals to treat a specific condition or group of conditions and labeled as such 610. Including the integrated enriched food products into a person's daily dietary menu is providing additional dietary elements with integrated enriched supplemental vitamins and minerals to provide a level of treatment for various conditions 620. The integrated enriched food products cost can be competitive with other food products 630. The cost to cultivate and deliver the integrated enriched supplemental vitamins and minerals food products will be far less than current healthcare delivery systems 640 of one embodiment.

Hydroponic Growing Container with Young Plant and Root System:

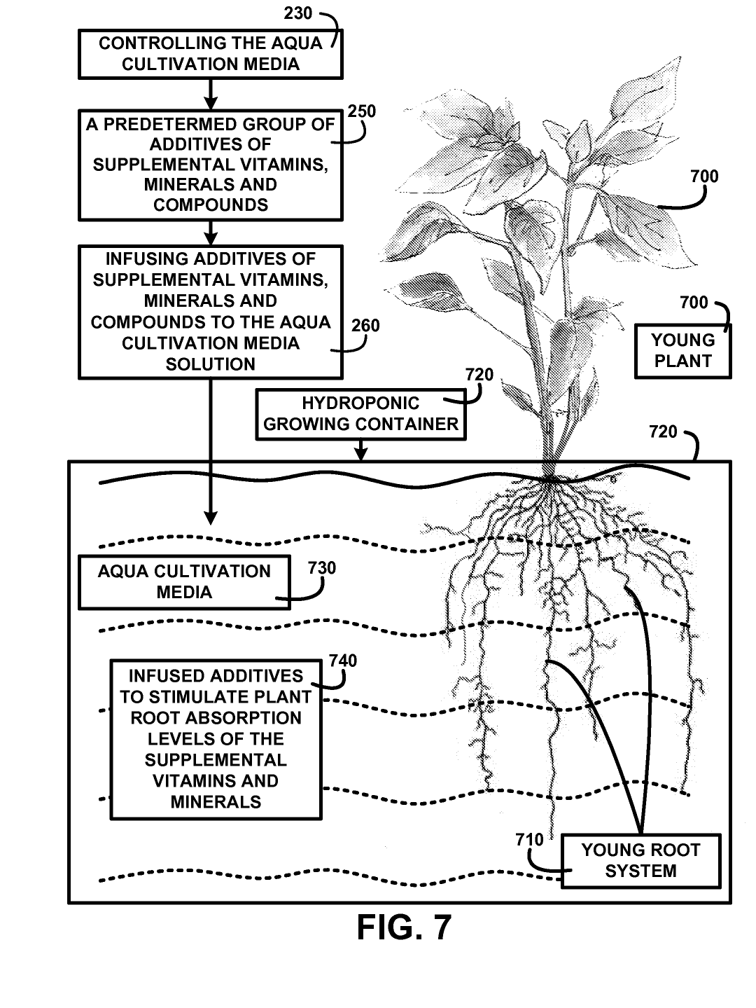

FIG. 7 shows for illustrative purposes only an example of hydroponic growing container with young plant and root system of one embodiment. FIG. 7 shows an example of the cultivation system used to cultivate integrated enriched food products 100 of FIG. 1 including the use of hydroponic cultivation to raise integrated enriched food products including fruits, produce and aquatic species and animal feed 110 of FIG. 1. An example of using hydroponic cultivation to raise integrated enriched food products includes controlling the aqua cultivation media 230 to include additives of supplemental vitamins and minerals 250 of one embodiment.

Minerals & compounds to be used in hydroponic/aqua solution 260 are absorbed by a young plant 700 through a young root system 710. The aqua cultivation media 730 is circulated in a hydroponic growing container 720 including adding additives to stimulate plant root absorption levels of the supplemental vitamins and minerals 740 of one embodiment.

### Developed “Fruit” Contains Supplemental Vitamins and Minerals:

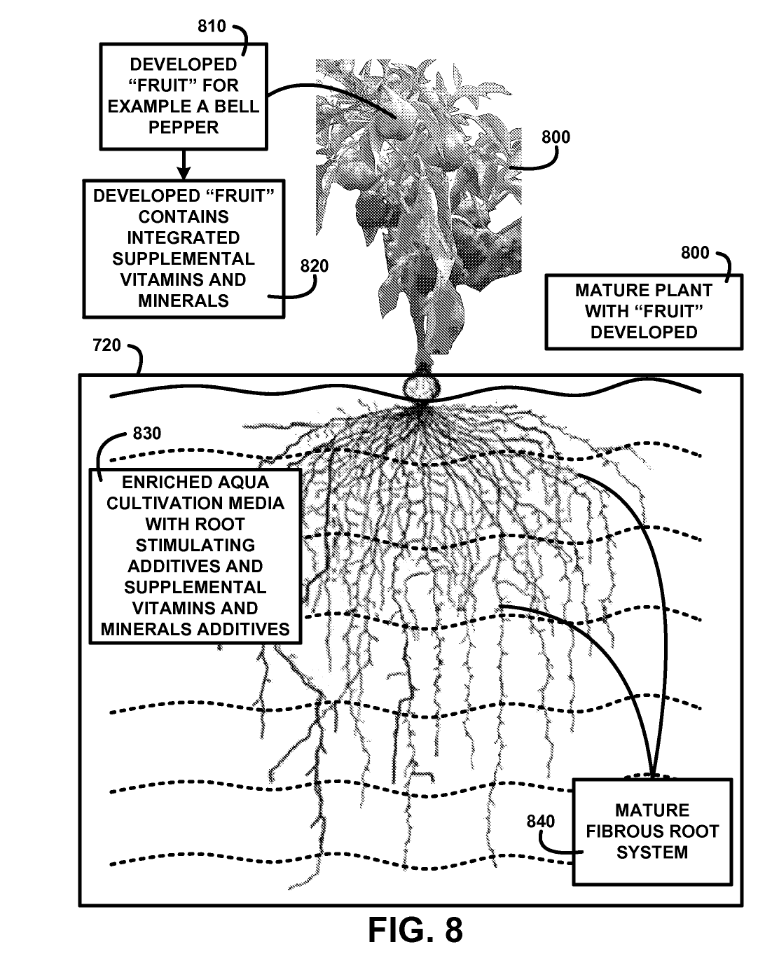

FIG. 8 shows for illustrative purposes only an example of developed “fruit” contains supplemental vitamins and minerals of one embodiment. FIG. 8 shows a mature plant with “fruit” developed 800. The mature plant can include any species or type of fruit, vegetable, grain or other plant and produce developed “fruit” for example a bell pepper 810. The developed “fruit” contains supplemental vitamins and minerals 820 integrated into the growth of the plant using for example a hydroponic growing container 720. In the hydroponic growing container 720 an aqua cultivation media with root stimulating additives and supplemental vitamins and minerals 830 is absorbed by a mature fibrous root system 840 and the supplemental vitamins and minerals are distributed throughout the plant including the developed “fruit” of one embodiment.

### Controlling the Aqua Cultivation Media Composition:

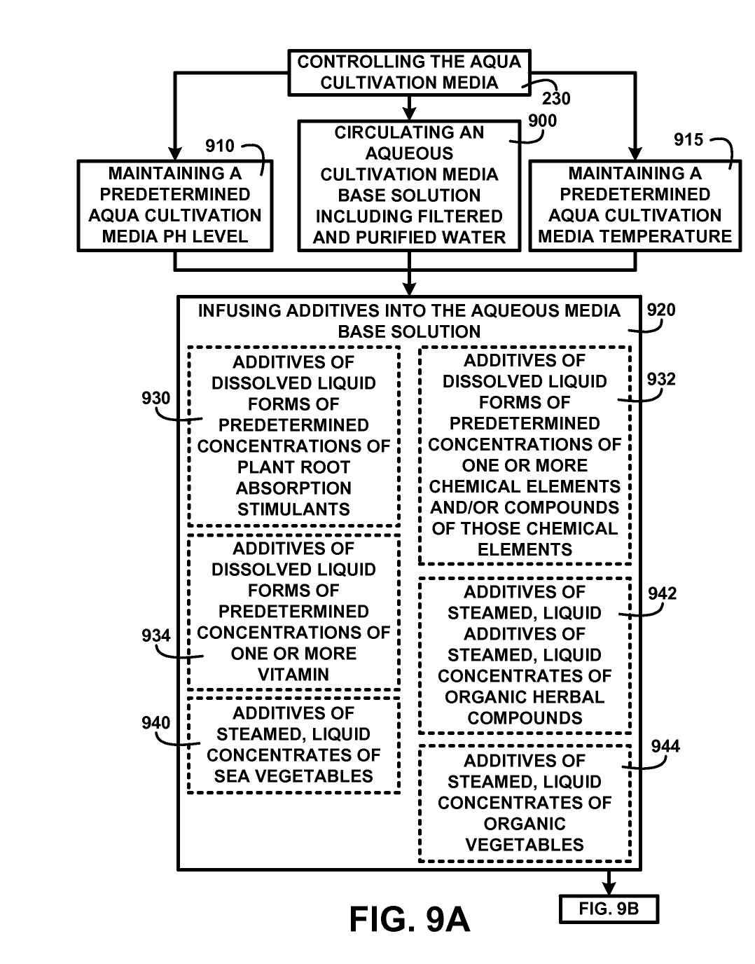

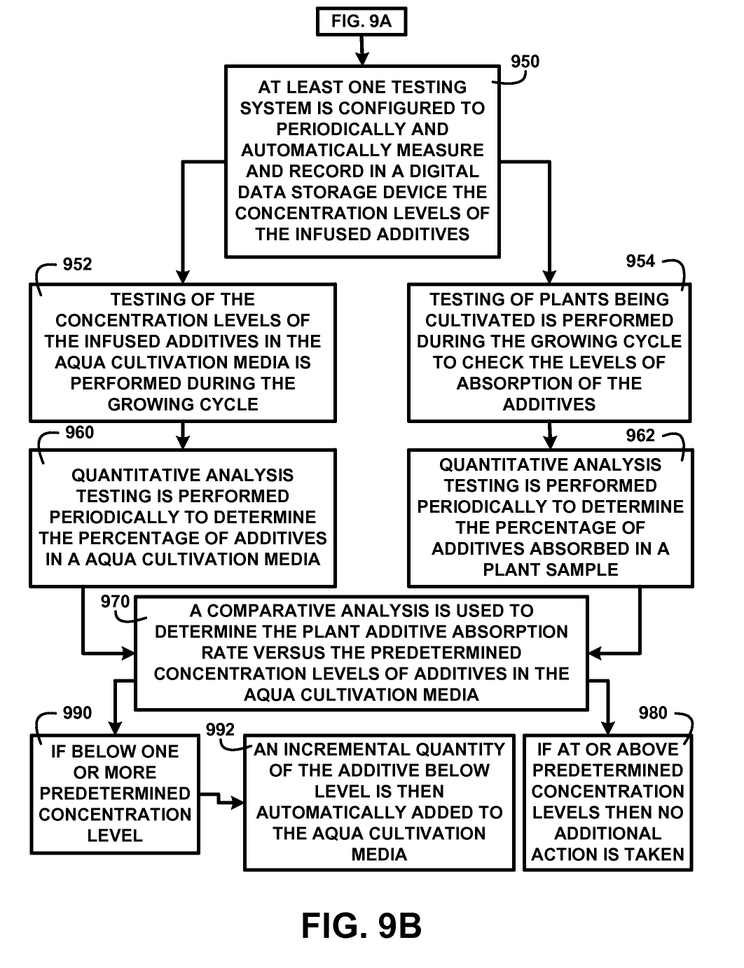

FIG. 9A shows a block diagram of controlling the aqua cultivation media composition of one embodiment. FIG. 9A shows controlling the aqua cultivation media composition 230 includes circulating an aqueous cultivation media base solution including filtered and purified water 900 in plant cultivation containers. Controlling the aqua cultivation media composition 230 includes maintaining a predetermined aqua cultivation media pH level 910. Controlling the aqua cultivation media composition 230 includes maintaining a predetermined aqua cultivation media temperature 915. Controlling the aqua cultivation media composition 230 includes infusing additives into the aqueous media base solution 920 including additives of dissolved liquid forms of predetermined concentrations of plant root absorption stimulants 930, additives of dissolved liquid forms of predetermined concentrations of one or more chemical elements and/or compounds of those chemical elements 932, additives of dissolved liquid forms of predetermined concentrations of one or more vitamin 934, additives of steamed, liquid concentrates of sea vegetables 940, additives of steamed, liquid additives of steamed, liquid concentrates of organic herbal compounds 942, and additives of steamed, liquid concentrates of organic vegetables 944 thereby enriching the aqua cultivation media of one embodiment. Controlling the aqua cultivation media composition 230 is further described in FIG. 9B.

### Testing the Aqua Cultivation Media Composition:

FIG. 9B shows a block diagram of testing the aqua cultivation media composition of one embodiment. FIG. 9B shows a continuation from FIG. 9A that includes testing the aqua cultivation media composition used for adjusting the predetermined concentration levels according to the plant species subject to cultivation using the aqua cultivation media and predetermined integrated concentrations to be absorbed through the root system of the subject plant species. At least one testing system is configured to periodically and automatically measure and record in a digital data storage device the concentration levels of the infused additives 950. Testing of the concentration levels of the infused additives in the aqua cultivation media is performed during the growing cycle 952. A quantitative analysis test is performed periodically to determine the percentage of additives in a aqua cultivation media 960. Testing of plants being cultivated is performed during the growing cycle to check the levels of absorption of the additives 954. A quantitative analysis test is performed periodically to determine the percentage of additives absorbed in a plant sample 962. A comparative analysis is used to determine the plant additive absorption rate versus the predetermined concentration levels of additives in the aqua cultivation media 970. Evaluation of the comparative analysis may show the plant additive absorption rate if at or above predetermined concentration levels then no additional action is taken 980. Evaluating the comparative analysis may show the plant additive absorption rate if below one or more predetermined concentration level 990, then an incremental quantity of the additive below level is then automatically added to the aqua cultivation media 992.

Chemical Elements and/or Compounds of Those Chemical Elements Additives:

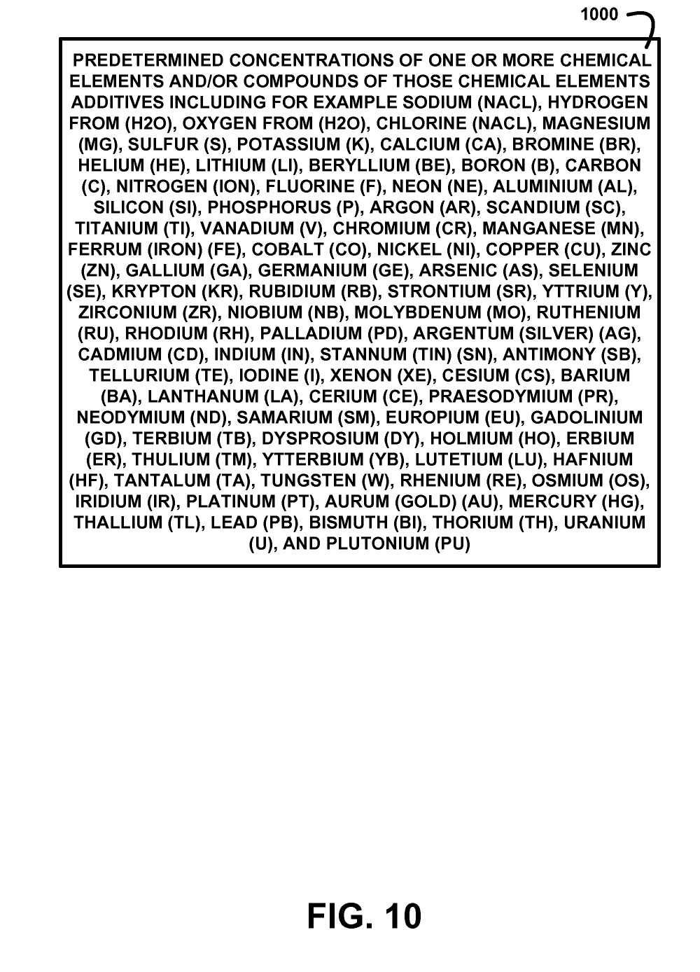

FIG. 10 shows a block diagram of examples chemical elements and/or compounds of those chemical elements additives of one embodiment. FIG. 10 shows examples of one or more chemical elements and/or compounds of those chemical elements additives infused into the aqua cultivation media in predetermined concentrations including for example Sodium (NaCl), Hydrogen from (H2O), Oxygen from (H2O), Chlorine (NaCl), Magnesium (Mg), Sulfur (S), Potassium (K), Calcium (Ca), Bromine (Br), Helium (He), Lithium (Li), Beryllium (Be), Boron (B), Carbon (C), Nitrogen (ion), Fluorine (F), Neon (Ne), Aluminium (Al), Silicon (Si), Phosphorus (P), Argon (Ar), Scandium (Sc), Titanium (Ti), Vanadium (V), Chromium (Cr), Manganese (Mn), Ferrum (Iron) (Fe), Cobalt (Co), Nickel (Ni), Copper (Cu), Zinc (Zn), Gallium (Ga), Germanium (Ge), Arsenic (As), Selenium (Se), Krypton (Kr), Rubidium (Rb), Strontium (Sr), Yttrium (Y), Zirconium (Zr), Niobium (Nb), Molybdenum (Mo), Ruthenium (Ru), Rhodium (Rh), Palladium (Pd), Argentum (silver) (Ag), Cadmium (Cd), Indium (In), Stannum (tin) (Sn), Antimony (Sb), Tellurium (Te), Iodine (I), Xenon (Xe), Cesium (Cs), Barium (Ba), Lanthanum (La), Cerium (Ce), Praesodymium (Pr), Neodymium (Nd), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Hafnium (Hf), Tantalum (Ta), Tungsten (W), Rhenium (Re), Osmium (Os), Iridium (Ir), Platinum (Pt), Aurum (gold) (Au), Mercury (Hg), Thallium (TI), Lead (Pb), Bismuth (Bi), Thorium (Th), Uranium (U), and Plutonium (Pu).

### Vitamin Liquid Additives:

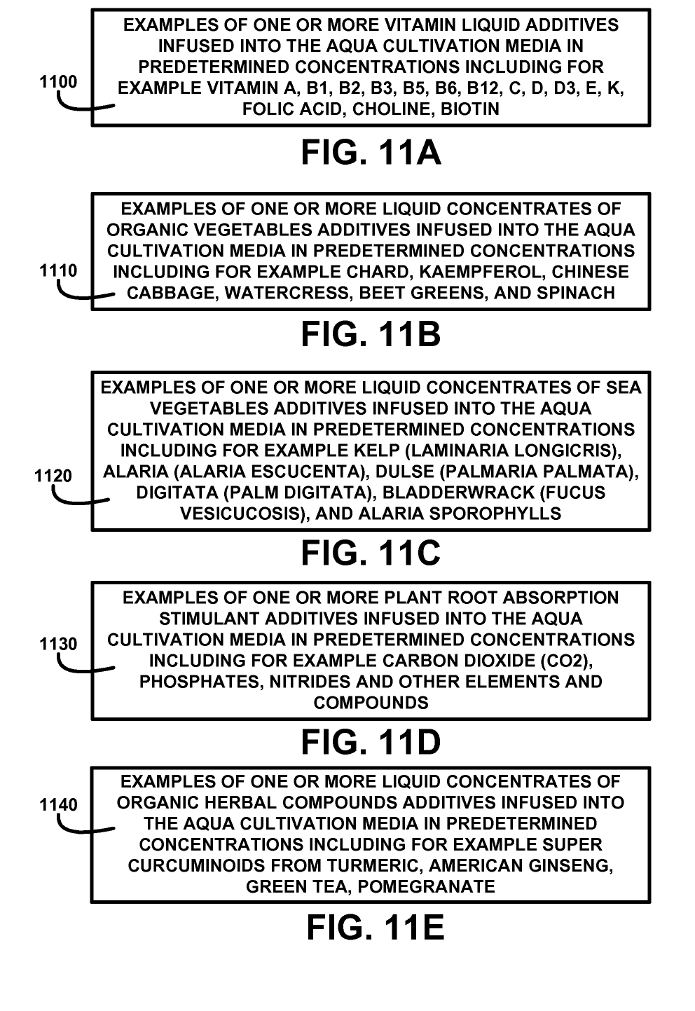

FIG. 11A shows a block diagram of examples of one or more vitamin liquid additives of one embodiment. FIG. 11A shows Examples of one or more vitamin liquid additives infused into the aqua cultivation media in predetermined concentrations including for example vitamin A, B1, B2, B3, B5, B6, B12, C, D, D3, E, K, Folic Acid, Choline, and Biotin 1100 of one embodiment.

### Organic Vegetables Additives:

FIG. 11B shows a block diagram of examples of one or more liquid concentrates of organic vegetables additives of one embodiment. FIG. 11B shows examples of one or more liquid concentrates of organic vegetables additives infused into the aqua cultivation media in predetermined concentrations including for example Chard, Kaempferol, Chinese Cabbage, Watercress, Beet Greens, and Spinach 1110 of one embodiment.

### Sea Vegetables Additives:

FIG. 11C shows a block diagram of examples of one or more liquid concentrates of sea vegetables additives of one embodiment. FIG. 11C shows examples of one or more liquid concentrates of sea vegetables additives infused into the aqua cultivation media in predetermined concentrations including for example Kelp (Laminaria longicris), Alaria (Alaria Escucenta), Dulse (Palmaria palmata), Digitata (Palm digitata), Bladderwrack (Fucus vesicucosis), and Alaria Sporophylls 1120 of one embodiment.

### Plant Root Absorption Stimulant Additives:

FIG. 11D shows a block diagram of examples of one or more plant root absorption stimulant additives of one embodiment. FIG. 11D shows examples of one or more plant root absorption stimulant additives infused into the aqua cultivation media in predetermined concentrations including for example Carbon Dioxide (CO2), phosphates, nitrides and other elements and compounds 1130 of one embodiment.

### Organic Herbal Compounds Additives:

FIG. 11E shows a block diagram of examples of one or more liquid concentrates of organic herbal compounds additives of one embodiment. FIG. 11E shows examples of one or more liquid concentrates of organic herbal compounds additives infused into the aqua cultivation media in predetermined concentrations including for example Super Curcuminoids from Turmeric, American Ginseng, Green Tea, and Pomegranate 1140 of one embodiment.

### Medicinal Herbs:

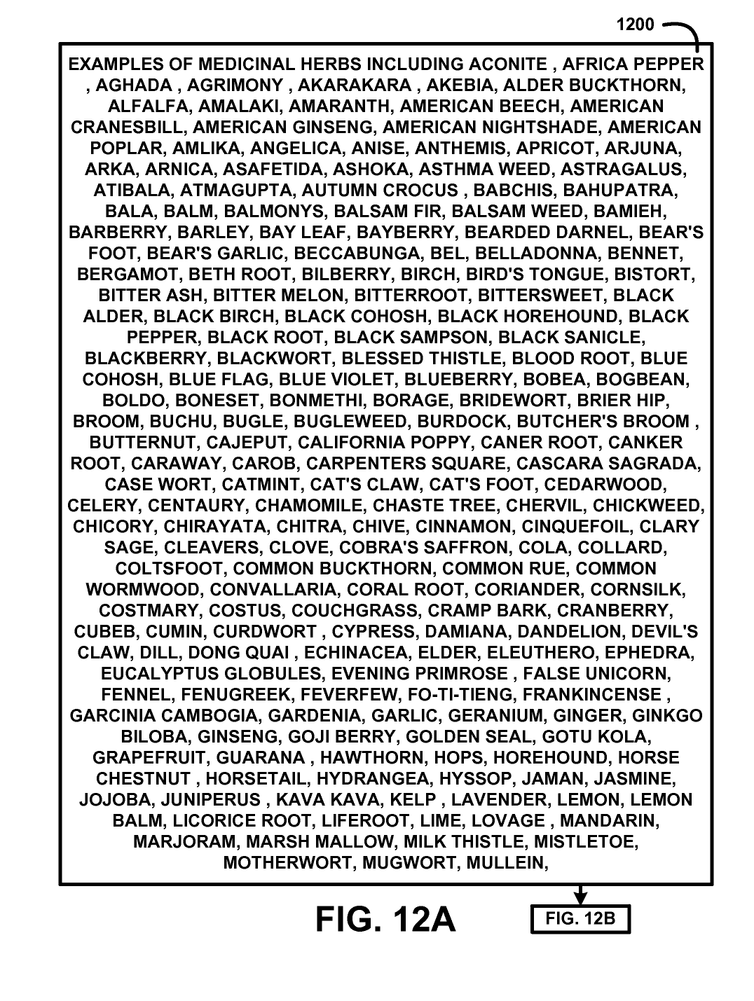

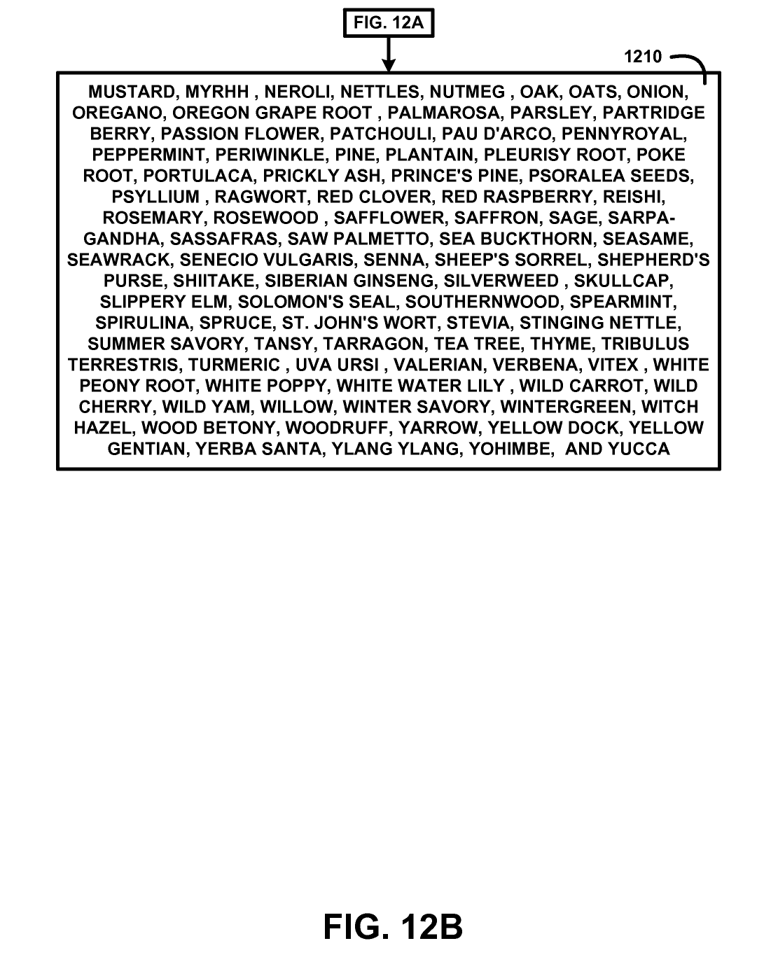

FIG. 12A shows a block diagram of examples of medicinal herbs of one embodiment. FIG. 12A shows examples of medicinal herbs including aconite, africa pepper, aghada, agrimony, akarakara, akebia, alder buckthorn, alfalfa, amalaki, amaranth, american beech, american cranesbill, american ginseng, american nightshade, american poplar, amlika, angelica, anise, anthemis, apricot, arjuna, arka, arnica, asafetida, ashoka, asthma weed, astragalus, atibala, atmagupta, autumn crocus, babchis, bahupatra, bala, balm, balmonys, balsam fir, balsam weed, bamieh, barberry, barley, bay leaf, bayberry, bearded darnel, bear's foot, bear's garlic, beccabunga, bel, belladonna, bennet, bergamot, beth root, bilberry, birch, bird's tongue, bistort, bitter ash, bitter melon, bitterroot, bittersweet, black alder, black birch, black cohosh, black horehound, black pepper, black root, black sampson, black sanicle, blackberry, blackwort, blessed thistle, blood root, blue cohosh, blue flag, blue violet, blueberry, bobea, bogbean, boldo, boneset, bonmethi, borage, bridewort, brier hip, broom, buchu, bugle, bugleweed, burdock, butcher's broom, butternut, cajeput, california poppy, caner root, canker root, caraway, carob, carpenters square, cascara sagrada, case wort, catmint, cat's claw, cat's foot, cedarwood, celery, centaury, chamomile, chaste tree, chervil, chickweed, chicory, chirayata, chitra, chive, cinnamon, cinquefoil, clary sage, cleavers, clove, cobra's saffron, cola, collard, coltsfoot, common buckthorn, common rue, common wormwood, convallaria, coral root, coriander, cornsilk, costmary, costus, couchgrass, cramp bark, cranberry, cubeb, cumin, curdwort, cypress, damiana, dandelion, devil's claw, dill, dong quai, echinacea, elder, eleuthero, ephedra, eucalyptus globules, evening primrose, false unicorn, fennel, fenugreek, feverfew, fo-ti-tieng, frankincense, garcinia cambogia, gardenia, garlic, geranium, ginger, ginkgo biloba, ginseng, goji berry, golden seal, gotu kola, grapefruit, guarana, hawthorn, hops, horehound, horse chestnut, horsetail, hydrangea, hyssop, jaman, jasmine, jojoba, juniperus, kava kava, kelp, lavender, lemon, lemon balm, licorice root, liferoot, lime, lovage, mandarin, marjoram, marsh mallow, milk thistle, mistletoe, motherwort, mugwort, mullein, 1200 of one embodiment. A continuation of examples of medicinal herbs is described in FIG. 12B.

### Continuation of Medicinal Herbs:

FIG. 12B shows a block diagram of a continuation of examples of medicinal herbs of one embodiment. FIG. 12B shows a continuation of examples of medicinal herbs including mustard, myrhh, neroli, nettles, nutmeg, oak, oats, onion, oregano, oregon grape root, palmarosa, parsley, partridge berry, passion flower, patchouli, pau d'arco, pennyroyal, peppermint, periwinkle, pine, plantain, pleurisy root, poke root, portulaca, prickly ash, prince's pine, psoralea seeds, psyllium, ragwort, red clover, red raspberry, reishi, rosemary, rosewood, safflower, saffron, sage, sarpa-gandha, sassafras, saw palmetto, sea buckthorn, seasame, seawrack, senecio vulgaris, senna, sheep's sorrel, shepherd's purse, shiitake, siberian ginseng, silverweed, skullcap, slippery elm, solomon's seal, southernwood, spearmint, spirulina, spruce, st. john's wort, stevia, stinging nettle, summer savory, tansy, tarragon, tea tree, thyme, tribulus terrestris, turmeric, uva ursi, valerian, verbena, vitex, white peony root, white poppy, white water lily, wild carrot, wild cherry, wild yam, willow, winter savory, wintergreen, witch hazel, wood betony, woodruff, yarrow, yellow dock, yellow gentian, yerba santa, ylang ylang, yohimbe, and yucca 1210 of one embodiment.

### Culinary Herbs and Spices:

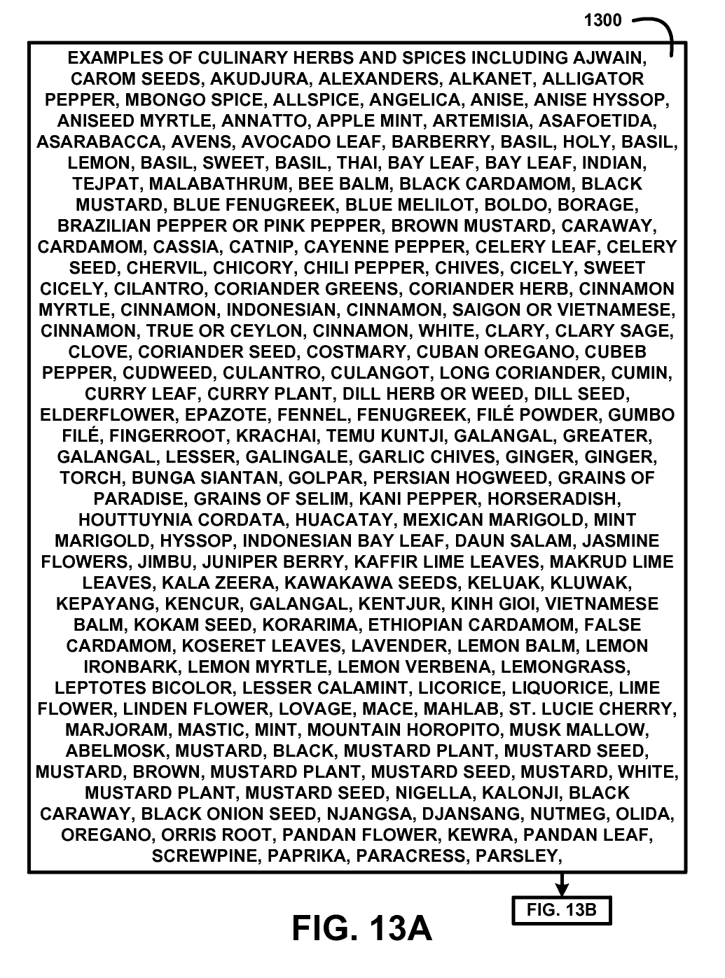

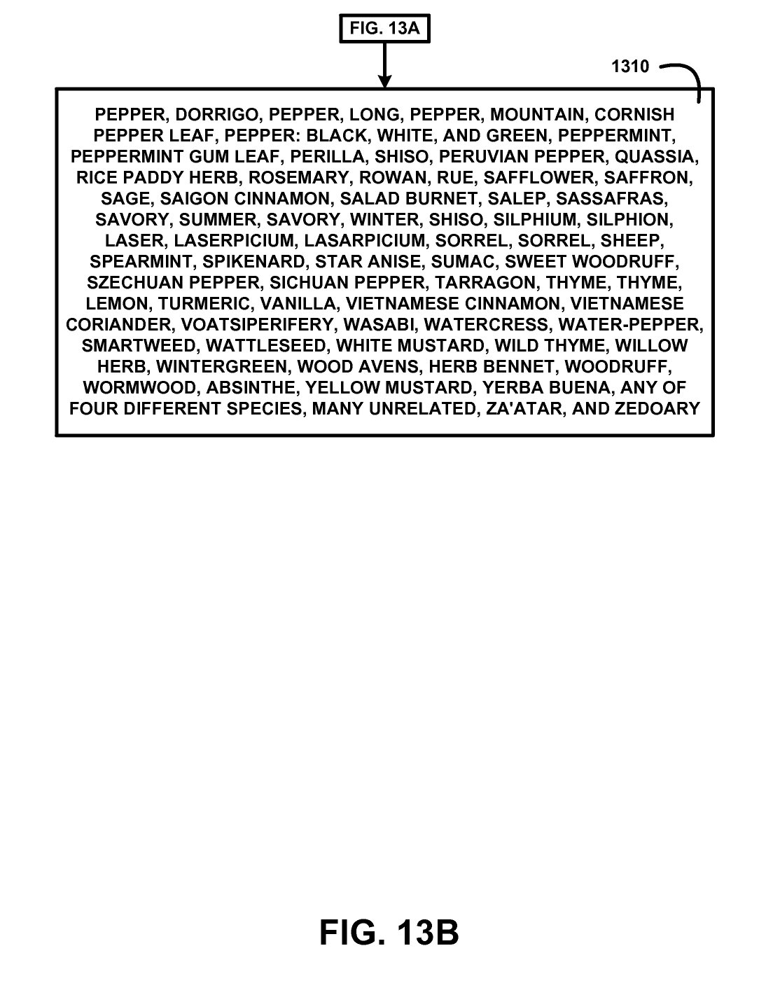

FIG. 13A shows a block diagram of examples of culinary herbs and spices of one embodiment. FIG. 13A shows examples of culinary herbs and spices including ajwain, carom seeds, akudjura, alexanders, alkanet, alligator pepper, mbongo spice, allspice, angelica, anise, anise hyssop, aniseed myrtle, annatto, apple mint, artemisia, asafoetida, asarabacca, avens, avocado leaf, barberry, basil, holy, basil, lemon, basil, sweet, basil, thai, bay leaf, bay leaf, indian, tejpat, malabathrum, bee balm, black cardamom, black mustard, blue fenugreek, blue melilot, boldo, borage, brazilian pepper or pink pepper, brown mustard, caraway, cardamom, cassia, catnip, cayenne pepper, celery leaf, celery seed, chervil, chicory, chili pepper, chives, cicely, sweet cicely, cilantro, coriander greens, coriander herb, cinnamon myrtle, cinnamon, indonesian, cinnamon, saigon or vietnamese, cinnamon, true or ceylon, cinnamon, white, clary, clary sage, clove, coriander seed, costmary, cuban oregano, cubeb pepper, cudweed, culantro, culangot, long coriander, cumin, curry leaf, curry plant, dill herb or weed, dill seed, elderflower, epazote, fennel, fenugreek, filé powder, gumbo filé, fingerroot, krachai, temu kuntji, galangal, greater, galangal, lesser, galingale, garlic chives, ginger, ginger, torch, bunga siantan, golpar, persian hogweed, grains of paradise, grains of selim, kani pepper, horseradish, houttuynia cordata, huacatay, mexican marigold, mint marigold, hyssop, indonesian bay leaf, daun salam, jasmine flowers, jimbu, juniper berry, kaffir lime leaves, makrud lime leaves, kala zeera, kawakawa seeds, keluak, kluwak, kepayang, kencur, galangal, kentjur, kinh gioi, vietnamese balm, kokam seed, korarima, ethiopian cardamom, false cardamom, koseret leaves, lavender, lemon balm, lemon ironbark, lemon myrtle, lemon verbena, lemongrass, leptotes bicolor, lesser calamint, licorice, liquorice, lime flower, linden flower, lovage, mace, mahlab, st. lucie cherry, marjoram, mastic, mint, mountain horopito, musk mallow, abelmosk, mustard, black, mustard plant, mustard seed, mustard, brown, mustard plant, mustard seed, mustard, white, mustard plant, mustard seed, nigella, kalonji, black caraway, black onion seed, njangsa, djansang, nutmeg, olida, oregano, orris root, pandan flower, kewra, pandan leaf, screwpine, paprika, paracress, parsley, 1300 of one embodiment.

### Continuation of Culinary Herbs and Spices:

FIG. 13B shows a block diagram of a continuation of examples of culinary herbs and spices of one embodiment. FIG. 13B shows a continuation of examples of culinary herbs and spices including pepper, dorrigo, pepper, long, pepper, mountain, cornish pepper leaf, pepper: black, white, and green, peppermint, peppermint gum leaf, perilla, shiso, peruvian pepper, quassia, rice paddy herb, rosemary, rowan, rue, safflower, saffron, sage, saigon cinnamon, salad burnet, salep, sassafras, savory, summer, savory, winter, shiso, silphium, silphion, laser, laserpicium, lasarpicium, sorrel, sorrel, sheep, spearmint, spikenard, star anise, sumac, sweet woodruff, szechuan pepper, sichuan pepper, tarragon, thyme, thyme, lemon, turmeric, vanilla, vietnamese cinnamon, vietnamese coriander, voatsiperifery, wasabi, watercress, water-pepper, smartweed, wattleseed, white mustard, wild thyme, willow herb, wintergreen, wood avens, herb bennet, woodruff, wormwood, absinthe, yellow mustard, yerba buena, any of four different species, many unrelated, za'atar, and zedoary 1310 of one embodiment.

### Grains:

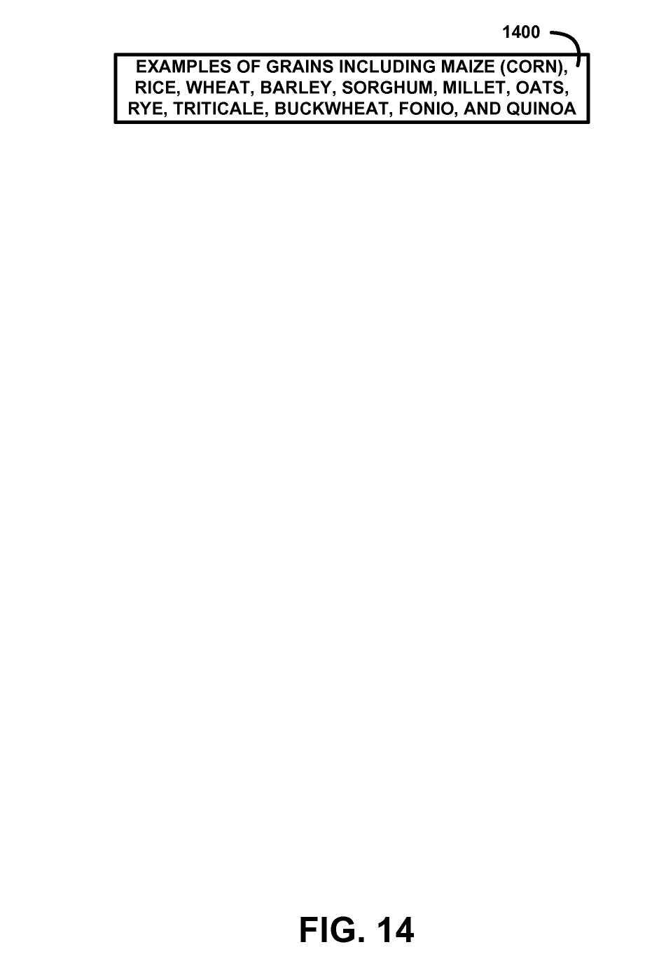

FIG. 14 shows a block diagram of examples of grains of one embodiment. FIG. 14 shows examples of grains including maize (corn), rice, wheat, barley, sorghum, millet, oats, rye, triticale, buckwheat, fonio, and quinoa 1400 of one embodiment.

The supplement and medication cultivated plant delivery system in one embodiment includes a supplement and medication cultivated plant delivery system, including using a cultivation system for cultivating integrated enriched food products including fruits, produce and aquatic species and animal feeds. In another embodiment the system includes controlling and testing the cultivation system environment including an aqua cultivation media. Other embodiments include incorporating a controlled feed system to raise aquatic species and animals using integrated enriched food products feeds. The system includes one embodiment for harvesting cultivated integrated enriched food products and cultivated integrated enriched food products feed raised aquatic species and animals. In yet another embodiment the system includes delivering the cultivated integrated enriched food stuffs to markets for providing alternative cultivated integrated enriched food stuffs to prevent and treat health conditions on a long-term sustainable basis, and empowering people to improve their health conditions while simply eating cultivated integrated enriched food stuffs in their daily regimen of eating. In one embodiment the system includes a cultivation system wherein the cultivation system may include hydroponic cultivation. In one embodiment the system includes controlling and testing the cultivation system environment including monitoring and adjusting an ambient temperature and humidity of the cultivation system environment including a cultivation building interior environment. In one embodiment the system includes controlling and testing the aqua cultivation media is achieved where predetermined concentrations of one or more elements and/or compounds of elements additives are added to the aqua cultivation media and concentration levels are tested during a plant growing cycle. In another embodiment the system includes predetermined concentrations of one or more elements and/or compounds of elements additives are predetermined according to a specific plant species subject to cultivation using the aqua cultivation media and predetermined integrated concentrations to be absorbed through the root system of the subject plant species. An embodiment includes controlling and testing the aqua cultivation media wherein predetermined concentrations of one or more vitamin additives are added to the aqua cultivation media and concentration levels are tested during a plant growing cycle. In yet another embodiment predetermined concentrations of one or more vitamin additives are predetermined according to a specific plant species subject to cultivation using the aqua cultivation media and predetermined integrated concentrations to be absorbed through the root system of the subject plant species. In one embodiment delivering the cultivated integrated enriched food stuffs to markets includes raw produce and fruits, packaged processed produce, fruits, aquatic species and animals and packaged prepared meals. In another embodiment the controlled feed system is configured to supply feed products to aquatic species and animals for daily consumption wherein the ingested integrated enriched food products feeds provide nutrition and supplemental minerals and vitamins to each organism. In one embodiment a cultivation system may include a cultivation environment that includes temperature and humidity control systems and pest and disease prevention control systems.

A supplement and medication cultivated plant delivery system in one embodiment includes a method, comprising cultivating integrated enriched food products including fruits, produce and aquatic species and animal feeds and introducing at least one mineral additive and at least one vitamin additive to a controlled cultivation environment including an aqua cultivation media for feeding fruits, produce and aquatic species and animal feeds an enriched aqua cultivation media to integrate the at least one mineral and at least one vitamin into specific plant species into the plant organism during the growing cycle. In one embodiment feeding aquatic species and animals with integrated enriched food products feeds to integrate the at least one mineral and at least one vitamin into the aquatic species and animals during a period of growth prior to harvest and slaughter. In another embodiment preparing harvested integrated enriched food products including fruits, produce and aquatic species and animal feeds is configured for delivery of integrated enriched food stuffs to markets for human consumption to prevent and treat health conditions on a long-term sustainable basis though a daily regimen of eating. In yet another embodiment cultivating integrated enriched food products is configured to include a cultivation system including hydroponic cultivation controlling and testing the cultivation system environment including monitoring and adjusting an ambient temperature and humidity of the cultivation system environment including a cultivation building interior environment. One embodiment introducing the at least one mineral additive and at least one vitamin additive in predetermined concentrations is configured wherein the predetermined concentrations are predetermined according to a specific plant species subject to cultivation using the enriched aqua cultivation media and predetermined integrated concentrations to be absorbed through the root system of the subject plant species. In another embodiment preparing harvested integrated enriched food products includes aquatic species and animals fed with integrated enriched food products feeds is configured to supply harvested and slaughtered aquatic species and animals for delivery of integrated enriched food stuffs to markets for human consumption to prevent and treat health conditions on a long-term sustainable basis though a daily regimen of eating. In yet another embodiment preparing harvested integrated enriched food products is configured to supply fruits and produce for delivery of integrated enriched food stuffs to markets for human consumption to prevent and treat health conditions on a long-term sustainable basis though a daily regimen of eating.

A supplement and medication cultivated plant delivery system in one embodiment includes an apparatus including at least one controlled cultivation environment including an aqua cultivation media to cultivate integrated enriched food products including fruits, produce and aquatic species and animal feeds. In another embodiment the apparatus includes at least one supply system to add at least one mineral and at least one vitamin in predetermined concentration levels into the aqua cultivation media to create an enriched aqua cultivation media to integrate the at least one mineral additive and at least one vitamin additive into specific plant species organism during the growing cycle to produce integrated enriched food products and feeds. One embodiment includes at least one testing system is configured to periodically and automatically measure and record in a digital data storage device the concentration levels of the at least one mineral additive and at least one vitamin additive in the enriched aqua cultivation media, and at least one controlled feed system to feed integrated enriched food products to aquatic species and animal while being raised. In one embodiment the integrated enriched food products are configured to produce integrated enriched food stuffs for delivery to markets for human consumption to prevent and treat health conditions on a long-term sustainable basis though a daily regimen of eating. In another embodiment the at least one supply system is configured to include automated controls of supply pumps and lines to add the at least one mineral and at least one vitamin in predetermined concentration levels into the aqua cultivation media to maintain the predetermined concentration levels. In another embodiment the at least one supply system is configured to supply predetermined concentrations of the at least one mineral additive and at least one vitamin additive wherein the predetermined concentrations are predetermined according to a specific plant species subject to cultivation using the enriched aqua cultivation media and predetermined integrated concentrations to be absorbed through the root system of the subject plant species. In one embodiment the at least one cultivation environmental control system is configured to include temperature and humidity control systems and pest and disease prevention control systems.

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

What is claimed is:

1. A supplement and medication cultivated plant delivery system, comprising:
using a cultivation system for cultivating integrated enriched food products including fruits, produce and aquatic species and animal feeds;
controlling and testing the cultivation system environment including an aqua cultivation media;
incorporating a controlled feed system to raise aquatic species and animals using integrated enriched food products feeds;
harvesting cultivated integrated enriched food products and cultivated integrated enriched food products feed raised aquatic species and animals;
delivering the cultivated integrated enriched food stuffs to markets for providing alternative cultivated integrated enriched food stuffs to prevent and treat health conditions on a long-term sustainable basis; and
empowering people to improve their health conditions while simply eating cultivated integrated enriched food stuffs in their daily regimen of eating.

2. The system of claim 1, wherein a cultivation system may include hydroponic cultivation.

3. The system of claim 1, wherein controlling and testing the cultivation system environment including monitoring and adjusting an ambient temperature and humidity of the cultivation system environment including a cultivation building interior environment.

4. The system of claim 1, wherein controlling and testing the aqua cultivation media is achieved where predetermined concentrations of one or more elements and/or compounds of elements additives are added to the aqua cultivation media and concentration levels are tested during a plant growing cycle.

5. The system of claim 4, wherein predetermined concentrations of one or more elements and/or compounds of elements additives are predetermined according to a specific plant species subject to cultivation using the aqua cultivation media and predetermined integrated concentrations to be absorbed through the root system of the subject plant species.

6. The system of claim 1, wherein controlling and testing the aqua cultivation media is achieved where predetermined concentrations of one or more vitamin additives are added to the aqua cultivation media and concentration levels are tested during a plant growing cycle.

7. The system of claim 6, wherein predetermined concentrations of one or more vitamin additives are predetermined according to a specific plant species subject to cultivation using the aqua cultivation media and predetermined integrated concentrations to be absorbed through the root system of the subject plant species.

8. The system of claim 1, wherein delivering the cultivated integrated enriched food stuffs to markets includes raw produce and fruits, packaged processed produce, fruits, aquatic species and animals and packaged prepared meals.

9. The system of claim 1, wherein the controlled feed system is configured to supply feed products to aquatic species and animals for daily consumption wherein the ingested integrated enriched food products feeds provide nutrition and supplemental minerals and vitamins to each organism.

10. The system of claim 1, wherein a cultivation system may include a cultivation environment that includes temperature and humidity control systems and pest and disease prevention control systems.

11. A method, comprising:
cultivating integrated enriched food products including fruits, produce and aquatic species and animal feeds;
introducing at least one mineral additive and at least one vitamin additive to a controlled cultivation environment including an aqua cultivation media for feeding fruits, produce and aquatic species and animal feeds an enriched aqua cultivation media to integrate the at least one mineral and at least one vitamin into specific plant species into the plant organism during the growing cycle;
feeding aquatic species and animals with integrated enriched food products feeds to integrate the at least one mineral and at least one vitamin into the aquatic species and animals during a period of growth prior to harvest and slaughter; and
preparing harvested integrated enriched food products including fruits, produce and aquatic species and animal feeds for delivery of integrated enriched food stuffs to markets for human consumption to prevent and treat health conditions on a long-term sustainable basis though a daily regimen of eating.

12. The method of claim 11, further comprising cultivating integrated enriched food products is configured to include a cultivation system including hydroponic cultivation controlling and testing the cultivation system environment including monitoring and adjusting an ambient temperature and humidity of the cultivation system environment including a cultivation building interior environment.

13. The method of claim 11, further comprising introducing the at least one mineral additive and at least one vitamin additive in predetermined concentrations wherein the predetermined concentrations are predetermined according to a specific plant species subject to cultivation using the enriched aqua cultivation media and predetermined integrated concentrations to be absorbed through the root system of the subject plant species.

14. The method of claim 11, wherein preparing harvested integrated enriched food products includes aquatic species and animals fed with integrated enriched food products feeds is configured to supply harvested and slaughtered aquatic species and animals for delivery of integrated enriched food stuffs to markets for human consumption to prevent and treat health conditions on a long-term sustainable basis though a daily regimen of eating.

15. The method of claim 11, wherein preparing harvested integrated enriched food products is configured to supply fruits and produce for delivery of integrated enriched food stuffs to markets for human consumption to prevent and treat health conditions on a long-term sustainable basis though a daily regimen of eating.

16. An apparatus, comprising:
at least one controlled cultivation environment including an aqua cultivation media to cultivate integrated enriched food products including fruits, produce and aquatic species and animal feeds;
at least one supply system to add at least one mineral and at least one vitamin in predetermined concentration levels into the aqua cultivation media to create an enriched aqua cultivation media to integrate the at least one mineral additive and at least one vitamin additive into specific plant species organism during the growing cycle to produce integrated enriched food products and feeds;
at least one testing system to periodically and automatically measure and record in a digital data storage device the concentration levels of the at least one mineral additive and at least one vitamin additive in the enriched aqua cultivation media; and
at least one controlled feed system to feed integrated enriched food products to aquatic species and animal while being raised.

17. The apparatus of claim 16, wherein the integrated enriched food products are configured to produce integrated enriched food stuffs for delivery to markets for human consumption to prevent and treat health conditions on a long-term sustainable basis though a daily regimen of eating.

18. The apparatus of claim 16, wherein the at least one supply system is configured to include automated controls of supply pumps and lines to add the at least one mineral and at least one vitamin in predetermined concentration levels into the aqua cultivation media to maintain the predetermined concentration levels.

19. The apparatus of claim 16, wherein the at least one supply system is configured to supply predetermined concentrations of the at least one mineral additive and at least one vitamin additive wherein the predetermined concentrations are predetermined according to a specific plant species subject to cultivation using the enriched aqua cultivation media and predetermined integrated concentrations to be absorbed through the root system of the subject plant species.

20. The apparatus of claim 16, wherein the at least one cultivation environmental control system is configured to include temperature and humidity control systems and pest and disease prevention control systems.
